(19) United States
(12) Patent Application Publication
(10) Pub. No.: US 2026/0265680 A1
(43) Pub. Date: Sep. 10, 2026

(54) A COMBINATION OF SUCCINATE CONSUMING MICROBIOTAS

(71) Applicant: TRANSLATIONAL HEALTH SCIENCE AND TECHNOLOGY INSTITUTE, Faridabad, Haryana (IN)

(72) Inventors: Amit AWASTHI, Faridabad (IN); Rajdeep DALAL, Faridabad (IN)

(21) Appl. No.: 19/489,180

(22) PCT Filed: May 28, 2024
(86) PCT No.: PCT/IN2024/050624

§ 371 Date: Dec. 1, 2025

(30) Foreign Application Priority Data
May 29, 2023 (IN) 202311037041

(51) Int. Cl. C12N 1/20; A61K 35/745; A61K 35/747; A61K 35/76; A61K 36/06; C12N 7/00; C12R 1/225

(52) U.S. Cl. CPC C12N 1/20; A61K 35/745; A61K 35/747; A61K 35/76; A61K 36/06; C12N 7/00; C12N 2740/10032; C12N 2795/00032; C12R 2001/225

(57) ABSTRACT

The present invention provides a combination of microbiotas selected from the group consisting of Phascolarctobacterium faecium, Phascolarctobacterium succinatutens, Dialister succinatutens, Dialister propionicifaciens, Bifidobacterium bifidum, and Lactobacillus intestinalis for its use in succinate consumption, a process for producing the said combination, a composition and utility thereof.

### FIELD OF THE INVENTION

The present invention relates to the field of Biotechnology. In particular, the present invention relates to a combination of commensal microbial consortia for its use in succinate consumption.

### BACKGROUND OF THE INVENTION

Microbiota-derived metabolites can act as a source of nutrients as well as a messenger molecule that can send a signal to distant organs to shape the host pathophysiology. In line with this, succinate is an example of such a metabolite, primarily, produced by gut commensal microbial cells including Bacteroides vulgatus and Bacteroides fragilis. The microbial dysbiosis is an inflammatory condition that led to overgrowth of the succinate-producing gut bacteria, which result to the accumulation of succinate within gut lumen. Increased accumulation of succinate aggravates the pathophysiology in inflammatory bowel diseases (IBD) and Clostridium difficile induced colitis. Recent reports have indicated that the elevated levels of succinate positively correlate with the pathophysiology associated with various disease conditions like obesity, cardiovascular disease (CVD), diabetes, non-alcoholic fatty liver disease (NAFLD), psoriasis, fibrosis (Liver and Lung), typhoid, and paratyphoid fever and other infections. Thus, the gut microbiota generates elevated levels of succinate that could be an indicator of tissue inflammation and potentially be targeted for the treatment or prevention of disease-associated pathophysiological conditions.

Succinate is an intermediate metabolite of the Tricarboxylic acid (TCA) cycle of host cell. However, succinate is also produced in large amounts during bacterial fermentation of dietary fiber. Recent studies indicate that succinate can activate immune cells via its specific surface receptor, SUCNR1/GPR91, and thus enhance inflammation [Nutrients. 2019 January; 11(1): 25.]. The increased succinate production can be due to the dysbiosis of gut commensal microbiota, which results in the overgrowth of the primary succinate producing commensal gut bacteria such as Bacteroides vulgatus and Bacteroides fragilis. Increased succinate production within the gut leads to its accumulation and ultimately aggravates the generation of inflammatory or pathogenic T cells. Therefore, the transplantation of consortia rich in succinate-consuming commensal gut bacteria together with microbiota, is considered to help in restoring colonization of succinate-consumers may limit tissue inflammation by restricting the availability of colonic succinate. Limiting the availability of colonic-succinate could decrease the generation and functions of inflammatory or pathogenic T cells associated with obesity, cardiovascular disease (CVD), Diabetes, NAFLD, typhoid, and paratyphoid fever and other intracellular bacterial infections, cancer, psoriasis (GV Pinget·2022; Cell Reports), Clostridium difficile-induced colitis, and IBD. Apparently, succinate-consuming consortia have the potential to produce propionate, which could be incredibly beneficial for individuals suffering from digestive ailments such as infantile colic. Additionally, these consortia may be useful in managing dysbiosis associated with colorectal cancer and cancer treatment, particularly allogeneic hematopoietic stem cell transplantation-associated diseases like Graft versus Host Disease (GvHD).

There are some compositions available in the prior art for instance:

US20160000838 discloses compositions comprising a single and specific Faecalibacterium prausnitzii strain, suitable e.g. in the treatment of inflammatory disorders, such as IBD without disclosing a mechanism of action.

WO2016086161 discloses compositions with increased abundance of commensal gut bacteria of the order Clostridiales, suitable in the treatment of GvHD (graft versus host disease).

WO2020079021 discloses compositions comprising specific consortia of living bacteria strains for treatment of intestinal dysbiosis related to cancer treatments, specifically allogeneic hematopoietic stem cell transplantation associated diseases, in particular Graft versus Host Disease (GvHD).

WO2020079024 discloses composition for treatment of colorectal cancer (CRC) and/or intestinal microbiome dysbiosis related to CRC treatment.

None of the prior art disclose use of microbial consortia that can reduce the succinate level and inhibit the pathology mediated by excessive succinate level. Therefore, there lies a requirement for a composition of a combination of commensal microbial consortia that are rich in succinate-consuming gut bacteria which allow the restoration of colonization of succinate-consuming gut bacteria to reduce the excessively produced succinate and succinate-induced pathophysiology and prevent disease-associated pathophysiological conditions such as obesity, cardiovascular disease (CVD), diabetes, Non-alcoholic fatty liver disease (NAFLD), typhoid, and paratyphoid fever, autoimmune and inflammatory diseases, cancer, intracellular infections, leaky gut associated with Alzheimer's, Parkinson, Huntington's, and other neurological disorders.

### OBJECT OF THE INVENTION

An object of the present invention is to provide a combination of commensal microbial consortia to reduce the succinate and succinate-induced pathophysiology, a process for producing the said combination, a composition and utility thereof.

### SUMMARY OF THE INVENTION

The present invention is drawn to a combination of succinate consuming microbiotas selected from the group consisting of Phascolarctobacterium faecium, Phascolarctobacterium succinatutens, Dialister succinatutens, Dialister propionicifaciens, Bifidobacterium bifidum, and Lactobacillus intestinalis in a ratio of 1:1:1:1:1:1 or 2:4:2:4:2:4 or 4:2:4:2:4:2. The present invention also discloses a composition comprising the combination along with pharmaceutically acceptable excipients.

The present invention is also drawn to a process of producing the combination of the present invention and its utility in reducing of excessively produced succinate and succinate-induced pathophysiology and preventing disease-associated pathophysiological conditions.

### BRIEF DESCRIPTION OF FIGURES

FIG. 1 depicts the individual strains grown on their respective culture plates.

FIG. 2 depicts a) Sensitized skin after topical application of 3% oxazolone; b) Inflammatory lesions present in colon structure after Intracolonic injection given to the sensitized animals; c) Histologic features present in the colon harvested from colitis and healthy controls.

FIG. 3 depicts a) Percentage change in body weight of animals treated with combination of microbial consortia; b) Inflammatory lesions formation in the colon harvested from the healthy, diseased and consortia treated animals; c) Histologic features present in the colon harvested from healthy control, diseased and consortia treated animals.

FIG. 4 depicts reduction in IL-9 m-RNA levels in CD4⁺ T cells harvested from Lamina propria.

FIG. 5 shows the reduction in foxol, major transcription factor for IL-9 secreting CD4⁺ T cells, m-RNA levels in CD4⁺ T cells harvested from Lamina propria.

FIG. 6 depicts reduction in SUCNR1/GPR91, a succinate specific receptor, m-RNA levels in CD4⁺ T cells harvested from Lamina propria.

FIG. 7 depicts the reduction in body weight of experimental animals treated with individual strains and a combination of consortia.

FIG. 8 depicts the gross pathology scores of experimental animals treated with individual strains and a combination of consortia.

FIG. 9 depicts the LCMS based measurement of stool succinate of experimental animals treated with individual strains and a combination of consortia.

FIG. 10 depicts the LCMS based measurement of stool propionate of experimental animals treated with individual strains and a combination of consortia.

FIG. 11 depicts a) Family belonging to major succinate consumers and associated members in experimental obese mice and wild type mice; b) Family belonging to major succinate producers and associated members in experimental obese mice and wild type mice.

FIG. 12 shows the following: a) The Regime that was followed for the therapeutic microbiota treatment in Db/Db mice; b) The Body weight gain score for the consortia treatment animals was found to be lower as compared to the untreated animals; c-g) The Glucose Tolerance test (IPGTT) was performed at different time points (10 Weeks, 12 Weeks, 14 Weeks, 16 Weeks & 18 Weeks) in Db/Db mice; h) The Serum succinate levels were measured using LC-MS in 18-week-old Db/Db mice.

### DETAILED DESCRIPTION OF THE PRESENT INVENTION

The present invention provides a combination comprising microbial consortia, a process for producing the said combination and utility of the combination.

The present invention provides a combination of succinate consuming microbiotas selected from the group consisting of Phascolarctobacterium faecium, Phascolarctobacterium succinatutens, Dialister succinatutens, Dialister propionicifaciens, Bifidobacterium bifidum, and Lactobacillus intestinalis in a ratio of 1:1:1:1:1:1 or 2:4:2:4:2:4 or 4:2:4:2:4:2.

A combination of succinate consuming microbiotas in a ratio of 1:1:1:1:1:1 has preferable values of Phascolarctobacterium faecium in the range of 1 mg/ml to 2 mg/ml, Phascolarctobacterium succinatutens in the range of 1 mg/ml to 2 mg/ml, Dialister succinatutens in the range of 1 mg/ml to 2 mg/ml, Dialister propionicifaciens in the range of 1 mg/ml to 2 mg/ml, Bifidobacterium bifidum in the range of 1 mg/ml to 2 mg/ml, Lactobacillus intestinalis in the range of 1 mg/ml to 2 mg/ml.

A combination of succinate consuming microbiotas in a ratio of 2:4:2:4:2:4 has preferable values of Phascolarctobacterium faecium in the range of 2 mg/ml to 4 mg/ml, Phascolarctobacterium succinatutens in the range of 4 mg/ml to 8 mg/ml, Dialister succinatutens in the range of 2 mg/ml to 4 mg/ml, Dialister propionicifaciens in the range of 4 mg/ml to 8 mg/ml, Bifidobacterium bifidum in the range of 2 mg/ml to 4 mg/ml, Lactobacillus intestinalis in the range of 4 mg/ml to 8 mg/ml.

A combination of succinate consuming microbiotas in a ratio of 4:2:4:2:4:2 has preferable values of Phascolarctobacterium faecium in the range of 4 mg/ml to 8 mg/ml, Phascolarctobacterium succinatutens in the range of 2 mg/ml to 4 mg/ml, Dialister succinatutens in the range of 4 mg/ml to 8 mg/ml, Dialister propionicifaciens in the range of 2 mg/ml to 4 mg/ml, Bifidobacterium bifidum in the range of 4 mg/ml to 8 mg/ml, Lactobacillus intestinalis in the range of 2 mg/ml to 4 mg/ml.

The present invention also discloses a composition comprising the combination along with pharmaceutically acceptable excipients that can be selected from the group comprising microcrystalline cellulose, mannitol, starch, maltodextrins, dextrins produced from non-GM sources.

The combination comprises microbiota Phascolarctobacterium faecium in the range of 1×10⁵ to 1×10¹⁰ CFU/ml, preferably 1×10⁷ to 1×10⁸ CFU/ml.

The combination comprises microbiota Phascolarctobacterium succinatutens in the range of 1×10⁵ to 1×10¹⁰ CFU/ml, preferably 1×10⁷ to 1×10⁸ CFU/ml.

The combination comprises microbiota Dialister succinatutens is in the range of 1×10⁵ to 1×10¹⁰ CFU/ml, preferably 1×10⁷ to 1×10⁸ CFU/ml.

The combination comprises microbiota Dialister propionicifaciens is in the range 1×10⁵ to 1×10¹⁰ CFU/ml, preferably 1×10⁷ to 1×10⁸ CFU/ml.

The combination comprises microbiota Bifidobacterium bifidum is in the range 1×10⁵ to 1×10¹⁰ CFU/ml, preferably 1×10⁷ to 1×10⁸ CFU/ml.

The combination comprises microbiota Lactobacillus intestinalis is in the range of 1×10⁵ to 1×10¹⁰ CFU/ml, preferably 1×10⁷ to 1×10⁸ CFU/ml.

The combination comprises equal of amount of each bacterial strain in the range of 1×10⁵ to 1×10¹⁰ CFU/ml, preferably 1×10⁷ to 1×10⁸ CFU/ml.

The biomass of individual species per dose present in the combination of a microbial consortium is detailed in Table A.

TABLE A
Biomass of individual species per dose

| Species | Consortia Biomass |
|---|---|
| Phascolarctobacterium faecium | 1 × 10⁷ to 5 × 10⁸ CFU/dose |
| Phascolarctobacterium | 1 × 10⁷ to 5 × 10⁸ CFU/dose |
| succinatutens | |
| Dialister succinatutens | 1 × 10⁷ to 5 × 10⁸ CFU/dose |
| Dialister propionicifaciens | 1 × 10⁷ to 5 × 10⁸ CFU/dose |
| Bifidobacterium bifidum | 1 × 10⁷ to 5 × 10⁸ CFU/dose |
| Lactobacillus intestinalis | 1 × 10⁷ to 5 × 10⁸ CFU/dose |

The combination comprises microbiotas that are present in form of spores, in-vitro cultures, L-dried (lyophilized) culture or freeze-dried microbial cell powder.

Optionally, the present invention discloses that the combination additionally consists of an endosymbiotic microbial consortium, an ectosymbiotic microbial consortia or combination of endosymbiotic or ectosymbiotic microbial consortia.

The endosymbiotic microbial consortia can be an oral or colonic (mucosal or fecal) microbiota that can be selected from the group comprising firmicutes, bacteroidetes, actinobacteria, proteobacteria, fusobacteria, and verrucomicrobia.

The ectosymbiotic microbial consortia can be a skin or vaginal microbiota, that can be selected from the group comprising firmicutes, bacteroidetes, actinobacteria, Proteobacteria, Fusobacteria, Verrucomicrobia and fungi belonging to genus Malassezia and Arthrodermataceae.

The combination comprises endosymbiotic microbial consortia is in the range of 1×10⁵ to 1×10⁹ CFU/ml, preferably 1×10⁷ to 1×10⁸ CFU/ml.

The combination comprises ectosymbiotic microbial consortia is in the range 1×10⁵ to 1×10⁹ CFU/ml, preferably 1×10⁷ to 1×10⁸ CFU/ml.

The microbial consortium modulates the number or/and functions of effector or inflammatory T cells (Th1, Th2, Th17, Th9, Th22, TFH) and regulatory T cells (Foxp3⁺ T cells, Tr1 cells, and RORγt⁺ T reg cells) or/and innate immune cells of mammals and preferably humans.

Optionally, the present invention discloses the combination additionally consists of a mycobiota, or a virobiota or a combination of mycobiota or virobiota.

The mycobiota can be selected from the group comprising Candida (Candida albicans, Candida tropicalis, Candida parapsilosis, Candida glabrata, Candida krusei, Candida lusitaniae etc) Saccharomyces (Saccharomyces cerevisiae), Penicillium (Penicillium commune), Aspergillus (Aspergillus versicolor), Cryptococcus, Malassezia (Malassezia globosa, Malassezia restricta, Malassezia pachydermatis), Cladosporium (Cladosporium herbarum), Galactomyces (Galactomyces geotrichum), Debaryomyces (Debaryomyces hansenii), and Trichosporon.

The virobiota can be selected from the group comprising human endogenous retroviruses (HERVs), gut bacteriophages naturally present in humans and mice that can be selected from the group comprising Caudaovirales, Alteromondales, Clostridiales, Phages belonging to acetobutylicum and retroviridae family, temperate phages, microbicidal phages, Phages infecting E. coli, Lactobacillus, and Bacteroides, Enterobacteria phage, Phages infecting Bacteroides uniforms and Bacteroides thetaiotaomicron, Clostridiales phages.

The combination comprises mycobiota is in the range of 10⁵ to 10⁸ CFU/ml, preferably 10⁷ to 10⁸ CFU/ml.

In an embodiment, the present invention discloses a composition, comprising the combination of microbiotas, along with pharmaceutically acceptable excipients that can be selected from the group comprising microcrystalline cellulose, mannitol, starch, maltodextrins, dextrins produced from non-GM sources.

The present invention discloses a composition, comprising the combination of microbiotas, present in a pharmaceutical acceptable carrier or matrix that can be selected from the group comprising kefiran, guar gum, duck feet gelatin (DFG), Dextrin, polydextrose, sago starch, Pectin, Chitosan, Chondroitin sulphate, Dextran, Cyclodextrin, Insulin, Xanthan gum, Amylose, Alginates, Shellac, Locust bean gum, methacrylic acid, methacrylic/acrylic esters, Polyvinyl acetate phthalate, Hydroxypropyl ethylcellulose phthalate, Cellulose acetate trimellitate, Cellulose acetate phthalate, hydroxypropyl methylcellulose acetate succinate (HPMCAS), and hydroxypropyl methyl-cellulose phthalate (HPMCP), azo-containing polymers and polyesters.

The azo-containing polymers can be selected from the group comprising azo-containing polyurethanes, azopolymeric hydrogels, copolymers of styrene, 2-hydroxyethyl methacrylate (HEMA) cross-linked with divinyl azobenzene (DVAB).

The polyesters can be selected from the group comprising poly D L-lactic-co-glycolic acid (PLGA), polyacrylamides, and polyvinyl alcohol (PVA).

In an embodiment, the present invention discloses a composition, comprising the combination of microbiotas, along with at least one non-digestible oligosaccharide that can be selected from the group consisting of Fructooligosaccharides (FOS), Pectins or pectic polysaccharides, Mannans, Pentosans, β-glucans, arabinans or galactans, Human Milk Oligosaacarides (HMOs), Glucooligosaccharide, Xylooligosaccharide, Galactooligosaccharide, Arabinoxylan, Arabinogalactan, Galactomannan, Polydextrose, Oligofructose, Inulin and mixtures or derivatives thereof.

In an embodiment, the present invention discloses a composition, comprising the combination of microbiotas, along with at least lactobacillus species that can be selected from the group comprising Lactobacillus acidophilus, Lactobacillus casei, Lactobacillus fermentum, Lactobacillus salivarius, Lactobacillus Intestinalis, Lactobacillus brevis, Lactobacillus leishmanii, Lactobacillus plantarum and Lactobacillus cellobiosus.

The composition comprising the combination of microbiotas can be microencapsulated using a technique such as spray-drying, freeze-drying, foam drying, and fluidized bed drying.

The composition can be administrated as oral, intradermal, transdermal, parenteral, intramuscular, intrathecal, topically, intravaginal, intravesical, intracisternal, or rectal. The composition can be in the form of solid, semi-solid, lyophilized powder, liquid dosage forms or aerosols i.e. the composition can be administrated in the form of Tablets, Capsules, granules, Drops, Sachets, Liquid Suspensions, emulsions, Chewable Tablets, and Capsules with Enteric Coating etc.

In an embodiment, the combination of the present invention can be prepared by a process containing the steps of:
(a) Site selection and sample collection:
   Lyophilized or L-dried cultures of each bacterium strain is selected from JCM, Microbe division, RIKEN BioResource Research Center, Japan.
(b) Isolation:
   Each strain of the bacterium is cultured in a broth as well as in a solid media using their respective culture medias in glove box chamber. Strict anaerobic environment is maintained for selective enrichment and isolation of each strain.
(c) Characterization:
   Characterization of each strain based on their morphology, gram staining, and sequencing using their specific primers or metagenomics.
(d) Preservation:
   The isolated strains are preserved in a Glycerol stock comprising 15-20% glycerol and 80-85% 1×PBS or NAPG Buffer and are stored at −80 to ~196° C. for future applications.
(e) Quantification:
   CFU per ml or gram is calculated by determining the serial dilutions for each bacterial strain.
(f) Development and optimization:
   Desired CFU for each strain is mixed to develop the consortia.

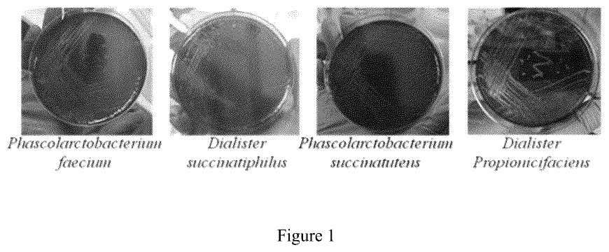

The L-type Pure strains of Phascolarctobacterium succinatutens JCM 16074, Phascolarctobacterium faecium JCM 30894, Dialister succinatiphilus JCM 15077, Dialister propionicifaciens JCM 17568, Bifidobacterium bifidum JCM 1254 and Lactobacillus intestinalis JCM 7548 were purchased from Microbe Division (RIKEN BRC; Japan). All above mentioned strains were handled in an anaerobic glove box and chamber containing anaerogas pack (Himedia; LE002A-5NO) and MGC oxygen indicator (Ageless Eye). Out of them Phascolarctobacterium succinatutens and Phascolarctobacterium faecium were cultured on Columbia blood agar (5% Sheep Blood) plates containing 1% succinate and simultaneously in Tryptic Soy Broth. Rest four bacteria Dialister succinatiphilus, Dialister propionicifaciens, Bifidobacterium bifidum JCM 1254 and Lactobacillus intestinalis JCM 7548 were cultured on Tryptic Soy blood agar as well as in Tryptic Soy Broth, simultaneously. Glycerol stocks of each bacterium were stored in-80 to ~196° C. for future uses. The culture plates depicting growth of succinate consuming microbiotas shown in FIG. 1.

In another embodiment, the combination and the composition containing the combination of the present invention reduces the succinate level and inhibit the pathology mediated by excessive succinate level, such as, microbial dysbiosis that can be selected from the group comprising obesity, cardiovascular disease (CVD), diabetes, Non-alcoholic fatty liver disease (NAFLD), typhoid, and paratyphoid fever, autoimmune and inflammatory diseases, cancer, intracellular infections, leaky gut associated with Alzheimer's, Parkinson, Huntington's, and other neurological disorders. Advantages of the present combination include:
Microbial consortia can be used as a nutraceutical that can be selected from the group comprising probiotics, post biotics, or both (Synbiotics), to treat or prevent the pathological conditions.
Microbial consortia can be used to improve the leaky gut associated with Alzheimer's, Parkinson, Huntington's, and other neurological disorders.
Microbial consortia can be used as a MIND diet (The Mediterranean-DASH Intervention for Neurodegenerative Delay diet) to improve the leaky gut associated with the pathological conditions.
Microbial consortia can be used with the selective blockers of metabolite sensing receptor of succinate (SUCNR1/GPR91) to treat or prevent the pathological conditions.
Microbial consortia can be used with antidiabetic drugs (Oral/Injectables) for the management of type 2 diabetes.

The present invention is illustrated by way of examples. The examples are meant only for illustrative purposes and are not meant to restrict the invention in any manner. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### COMBINATION OF THE PRESENT INVENTION

### Example 1: Establishment of an Experimental Colitis Model to Study the Effect of Microbial Consortia

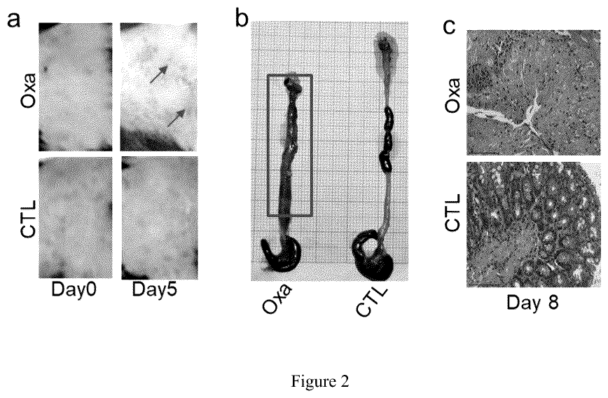

For studying the effect of microbial consortia an oxazolone-induced colitis was performed in a mice, as this model's macroscopic and microscopic histologic features are highly reminiscent of human inflammatory bowel disease, mainly Ulcerative colitis (UC). For the induction of acute colitis by oxazolone, wild-type mice (6 to 8 weeks) were sensitized by epicutaneous application of 100 μl of 3% of oxazolone (4-ethoxymethylene-2 phenyl-2-Oxazolin-5-one; Sigma-Aldrich; #SLCC3834) at a dilution of 4:1 in a mixture of acetone and oil (Olive oil; Del Monte) on day 0, followed by intrarectal (BECTON DICKINSON 427411 Intramedic Semi-Rigid Tubing, 0.023″ ID, 0.038″ OD, PE #50, Non-Sterile, 1825 Days Shelf Life) administration 100 μl of 0.5% oxazolone in 50% ethanol (EMSURE® ACS; Merck) on either day 6 or 7. Physical and pathological features present in the colon harvested from the colitis and healthy controls depicted in FIG. 2.

### Example 2: Colonization of Microbial Consortia Comprises Strain and its Effect on Colitis Progression

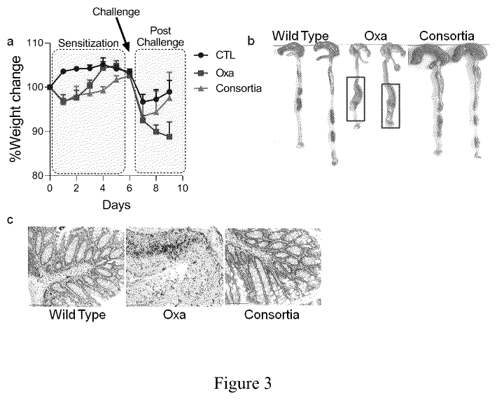

A number of desired CFU (~10⁷ to 10⁸ cfu/animal) of these strains was calculated based on OD measurements (0.4 to 0.5 OD) followed by culturing at different dilutions on their respective plates. Once the CFU calculations over, desired CFU of succinate-consuming and RA producing bacteria were mixed to form a desired consortia and gavaged in PEG-3350 treated animals. Later, recipient animals were pre-sensitized with 3% Oxazolone followed by Intracolonic challenge with 0.5% oxazolone to induce colitis and compare the disease severity in wild-type and animals colonized with microbial consortia, as depicted in FIG. 3.

### Example 3: Overall Disease Activity Score for Colitis Indicating Synergistic Response of Therapeutic Microbiota

To study the synergistic mechanism of action of a microbial consortium, the impact of individual strain and consortia compromises of all strains on colitis attributes was investigated. While studying the phenomenon of synergism, the overall disease activity of the colon harvested from colitis animals treated with individual strains and consortia was measured.

3.1. Reduction in IL-9 m-RNA Levels in CD4⁺ T Cells Harvested from Lamina Propria.

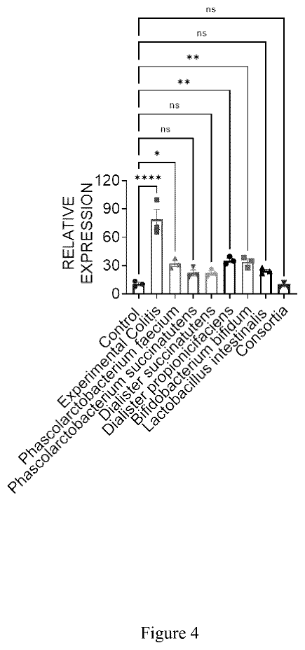

Experimental colitis, which mimics human inflammatory bowel diseases, was induced using the Oxazolone method. The probiotic formulations were administered orally in specific ratios via oral gavage. The animals were sacrificed at designated time points post-treatment. The colonic tissues were collected, and the lamina propria region was isolated using a digestion buffer followed by Percoll density gradient separation. Purified CD4⁺ T cells were isolated from the lamina propria mononuclear cells (LPMCs) using fluorescence-activated cell sorting (FACS). Total RNA was extracted from the isolated CD4⁺ T cells using a commercial RNA extraction kit. Reverse transcription (RT) was performed to convert RNA into complementary DNA (cDNA) using a reverse transcriptase enzyme and oligo (dT) primers. Specific primers for IL-9 and housekeeping genes (e.g., GAPDH/B-actin) were designed and validated using bioinformatics tools. Quantitative real-time PCR (qPCR) was performed using the synthesized cDNA as a template and specific primers to quantify IL-9 mRNA levels after normalization with the internal control (housekeeping gene). The data was then analyzed to check the relative expression of IL-9 after normalization with the internal control (housekeeping gene). The results are depicted in FIG. 4.

TABLE 1
Shows the qPCR-based expression of IL-9 in CD4+ T cells harvested from the Lamina
Propria (LP) of experimental animals treated with individual strains and a combination of consortia.
Reduction in IL-9 m-RNA levels in CD4⁺ T cells harvested from LPs

| | | | | Reduction of IL-9 | Reduction of IL-9 |
|---|---|---|---|---|---|
| | | IL-9 m-RNA | IL-9 m-RNA | expression with | expression with |
| | | reduction with | Reduction with | Dialister | Bifidobacterium |
| Disease | | Phascolarctobacterium | Phascolarctobacterium | succinatiphilus | bifidum treatment |
| Activity | IL-9 m-RNA | succinatutens treatment | faecium treatment vs | Treatment vs | vs Untreated |
| Score (DAS) | Reduction | vs Untreated animals | Untreated animals | Untreated animals | animals |
| 1 | >15% | | | ✓ | |
| 2 | 10-15% | ✓ | ✓ | | ✓ |
| 3 | 5-10% | | | | |
| 4 | 1-5% | | | | |

| | Reduction of IL-9 | | |
|---|---|---|---|
| | expression with | Reduction of IL-9 | |
| | Dialister | expression with | Reduction of IL-9 |
| Disease | propionicifaciens | Lactobacillus | expression with |
| Activity | treatment vs. | intestinalis treatment | Consortia treatment |
| Score (DAS) | Untreated animals | vs Untreated animals | vs Untreated animals |
| 1 | | | ✓ |
| 2 | ✓ | ✓ | |
| 3 | | | |
| 4 | | | |

3.2. Reduction in Foxo1, Major Transcription Factor for IL-9 Secreting CD4⁺ T Cells, m-RNA levels in CD4⁺ T cells harvested from Lamina propria.

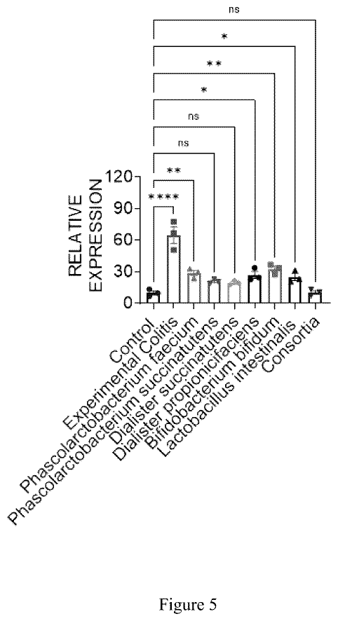

Experimental colitis, which mimics human inflammatory bowel diseases, was induced using the Oxazolone method. The probiotic formulations were administered orally in specific ratios via oral gavage. The animals were sacrificed at designated time points post-treatment. The colonic tissues were collected, and the lamina propria region was isolated using a digestion buffer followed by Percoll density gradient separation. Purified CD4⁺ T cells were isolated from the lamina propria mononuclear cells (LPMCs) using fluorescence-activated cell sorting (FACS). Total RNA was extracted from the isolated CD4⁺ T cells using a commercial RNA extraction kit. Reverse transcription (RT) was performed to convert RNA into complementary DNA (cDNA) using a reverse transcriptase enzyme and oligo (dT) primers. Specific primers for foxol and housekeeping genes (e.g., GAPDH/B-Actin) were designed and validated using bioinformatics tools. Quantitative real-time PCR (qPCR) was performed using the synthesized cDNA as a template and specific primers to quantify foxol mRNA levels after normalization with the internal control (housekeeping gene). The data was then analyzed to check the relative expression of foxol after normalization with the internal control (housekeeping gene). The results are depicted in FIG. 5.

TABLE 2
Shows the qPCR-based expression of Foxo1 in CD4⁺ T cells harvested from the Lamina
Propria (LP) of experimental animals treated with individual strains and a combination of consortia.
Reduction in foxo1 m-RNA levels in CD4⁺ T cells harvested from LPs

| | | | | Foxo1 m-RNA | Foxo1 m-RNA |
|---|---|---|---|---|---|
| | | Foxo1 m-RNA | Foxo1 m-RNA | Reduction with | Reduction with |
| | | reduction with | Reduction with | Dialister | Bifidobacterium |
| Disease | | Phascolarctobacterium | Phascolarctobacterium | succinatiphilus | bifidum treatment |
| Activity | Foxo1 m-RNA | succinatutens treatment | faecium treatment vs | Treatment vs | vs Untreated |
| Score (DAS) | Reduction | vs Untreated animals | Untreated animals | Untreated animals | animals |
| 1 | >15% | | | ✓ | |
| 2 | 10-15% | ✓ | ✓ | | ✓ |
| 3 | 5-10% | | | | |
| 4 | 1-5% | | | | |

| | Foxo1 m-RNA | | |
|---|---|---|---|
| | Reduction with | Foxo1 m-RNA | |
| | Dialister | Reduction with | Foxo1 m-RNA |
| Disease | propionicifaciens | Lactobacillus | Reduction with |
| Activity | treatment vs. | intestinalis treatment | Consortia treatment |
| Score (DAS) | Untreated animals | vs Untreated animals | vs Untreated animals |
| 1 | | | ✓ |
| 2 | ✓ | ✓ | |
| 3 | | | |
| 4 | | | |

3.3. Reduction in SUCNR1/GPR91, a Succinate Specific Receptor, m-RNA Levels in CD4⁺ T Cells Harvested from Lamina Propria.

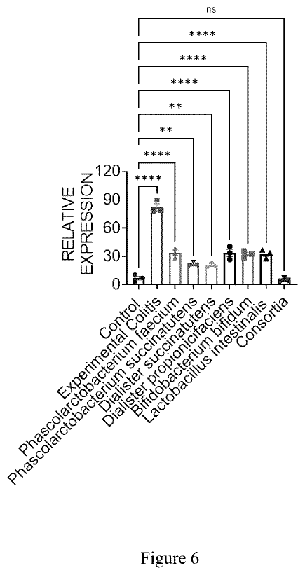

Experimental colitis, which mimics human inflammatory bowel diseases, was induced using the Oxazolone method. The probiotic formulations were administered orally in specific ratios via oral gavage. The animals were sacrificed at designated time points post-treatment. The colonic tissues were collected, and the lamina propria region was isolated using a digestion buffer followed by Percoll density gradient separation. Purified CD4⁺ T cells were isolated from the lamina propria mononuclear cells (LPMCs) using fluorescence-activated cell sorting (FACS). Total RNA was extracted from the isolated CD4⁺ T cells using a commercial RNA extraction kit. Reverse transcription (RT) was performed to convert RNA into complementary DNA (cDNA) using a reverse transcriptase enzyme and oligo (dT) primers. Specific primers for SUCNR1 and housekeeping genes (e.g., GAPDH/B-Actin) were designed and validated using bioinformatics tools. Quantitative real-time PCR (qPCR) was performed using the synthesized cDNA as a template and specific primers to quantify SUCNR1 mRNA levels after normalization with the internal control (housekeeping gene). The data was then analyzed to check the relative expression of SUCNR1 after normalization with the internal control (housekeeping gene). The results are depicted in FIG. 6.

TABLE 3
Shows the qPCR-based expression of Succinate receptor (SUCNR1/GPR91) in CD4⁺ T
cells harvested from the Lamina Propria (LP) of experimental animals
treated with individual strains and a combination of consortia.
Reduction in SUCNR1 (GPR91) m-RNA levels in CD4⁺ T cells harvested from LPs

| | | | | SUCNR1 m-RNA | SUCNR1 m-RNA |
|---|---|---|---|---|---|
| | | SUCNR1 m-RNA | SUCNR1 m-RNA | Reduction with | Reduction with |
| | | reduction with | Reduction with | Dialister | Bifidobacterium |
| Disease | | Phascolarctobacterium | Phascolarctobacterium | succinatiphilus | bifidum treatment |
| Activity | SUCNR1 m-RNA | succinatutens treatment | faecium treatment vs | Treatment vs | vs Untreated |
| Score (DAS) | Reduction | vs Untreated animals | Untreated animals | Untreated animals | animals |
| 1 | >15% | | | ✓ | |
| 2 | 10-15% | ✓ | ✓ | | ✓ |
| 3 | 5-10% | | | | |
| 4 | 1-5% | | | | |

| | SUCNR1 m-RNA | | |
|---|---|---|---|
| | Reduction with | SUCNR1 m-RNA | |
| | Dialister | Reduction with | SUCNR1 m-RNA |
| Disease | propionicifaciens | Lactobacillus | Reduction with |
| Activity | treatment vs. | intestinalis treatment | Consortia treatment |
| Score (DAS) | Untreated animals | vs Untreated animals | vs Untreated animals |
| 1 | | | ✓ |
| 2 | ✓ | ✓ | |
| 3 | | | |
| 4 | | | |

3.4. The Reduction in Body Weight of Experimental Animals Treated with Individual Strains and a Combination of Consortia.

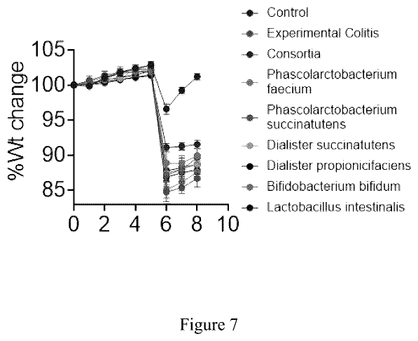

In this study, experimental colitis, which mimics human inflammatory bowel diseases, was induced using the Oxazolone method in animal models. Probiotic formulations were administered orally to the animals in specific ratios via gavage. Throughout the experiment, the body weight of each animal group was meticulously measured daily. This parameter served as a crucial indicator for assessing the severity of colitis among the different groups. By monitoring the reduction in body weight, the efficacy of the probiotic treatments in alleviating the symptoms of experimental colitis is compared. The results are depicted in FIG. 7.

TABLE 4
Shows the reduction in body weight of experimental animals treated
with individual strains and a combination of consortia.
Reduction in Body Weight

| | | | | Weight loss with | Weight loss with |
|---|---|---|---|---|---|
| | | Weight loss with | Weight loss with | Dialister | Bifidobacterium |
| Disease | | Phascolarctobacterium | Phascolarctobacterium | succinatiphilus | bifidum treatment |
| Activity | | succinatutens treatment | faecium treatment vs | Treatment vs | vs Untreated |
| Score (DAS) | Weight loss | vs Untreated animals | Untreated animals | Untreated animals | animals |
| 1 | 1-5% | | | | |
| 2 | 5-10% | ✓ | ✓ | ✓ | ✓ |
| 3 | 10-15% | | | | |
| 4 | >15% | | | | |

| | Weight loss with | | |
|---|---|---|---|
| | Dialister | Weight loss with | |
| Disease | propionicifaciens | Lactobacillus | Weight with |
| Activity | treatment vs | intestinalis treatment | Consortia treatment |
| Score (DAS) | Untreated animals | vs Untreated animals | vs Untreated animals |
| 1 | | | ✓ |
| 2 | ✓ | ✓ | |
| 3 | | | |
| 4 | | | |

3.5. The Gross Pathology Scores of Experimental Animals Treated with Individual Strains and a Combination of Consortia.

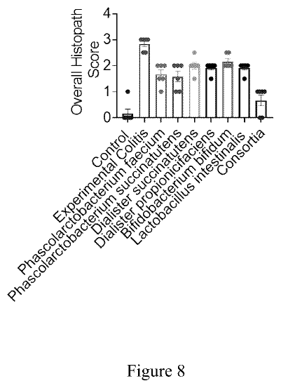

After sacrificing the animals, the colon tissue is fixed in formalin and processed for paraffin embedding. Thin sections are then cut from the paraffin-embedded tissue and stained with hematoxylin and eosin (H&E) for microscopic examination. Under the microscope, specific features of colitis, such as epithelial damage, inflammatory cell infiltration, goblet cell depletion, crypt distortion, and ulceration, are evaluated. These features are scored based on predetermined criteria, typically using a numerical scale ranging from 0 to 3 or 0 to 4 to indicate the severity of each parameter. Blinded observers independently assess the tissue sections, and the scores for individual criteria are summed to obtain a total histopathological score for each animal. Statistical analysis is then performed to compare the severity of colitis between different experimental groups. Histopathological scoring provides valuable insights into the effectiveness of treatments in mitigating colitis severity, contributing to our understanding of disease pathogenesis and therapeutic interventions. The results are depicted in FIG. 8.

TABLE 5
Shows the gross pathology scores of experimental animals
treated with individual strains and a combination of consortia.
Gross Pathology

| Gross | Phascolarctobacterium | Phascolarctobacterium | Dialister succinatiphilus | Bifidobacterium |
|---|---|---|---|---|
| Pathology | succinatutens treatment vs | faecium treatment vs | treatment vs | bifidum treatment vs |
| Score | Untreated animals | Untreated animals | Untreated animals | Untreated animals |
| 1 | | | ✓ | |
| 2 | ✓ | ✓ | | ✓ |
| 3 | | | | |

| | Dialister | | |
|---|---|---|---|
| Gross | propionicifaciens | Lactobacillus | |
| Pathology | treatment vs | intestinalis treatment | Consortia treatment |
| Score | Untreated animals | vs Untreated animals | vs Untreated animals |
| 1 | | | ✓ |
| 2 | ✓ | ✓ | |
| 3 | | | |

3.6. The LCMS Based Measurement of Stool Succinate of Experimental Animals Treated with Individual Strains and a Combination of Consortia.

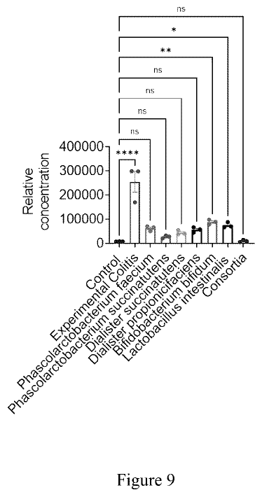

The stool samples were weighed and then homogenized in an extraction solvent to determine stool succinate levels using LC-MS. The supernatant was collected after centrifugation to remove insoluble debris. The LC-MS system was set up with a compatible column and mobile phase to separate and detect succinate. Calibration standards covering the expected range of succinate concentrations were prepared and used to generate a calibration curve by injecting them into the LC-MS system. Stool extracts were then injected into the LC-MS system, and the mass spectrometer separated and monitored succinate peaks. Quantification of succinate in the stool sample was achieved by comparing its peak area to the calibration curve. Quality control measures were implemented to ensure the accuracy and precision of the analysis. Data analysis involved integrating succinate peaks, calculating concentrations, and validating the method's performance. Ultimately, results were reported, providing valuable insights into stool succinate levels and their potential implications in physiological and pathological processes. The results are depicted in FIG. 9.

TABLE 6
Shows the LCMS based measurement of stool succinate of experimental animals
treated with individual strains and a combination of consortia.
Stool Succinate Reduction

| | | | | | Reduction with |
|---|---|---|---|---|---|
| | | Reduction with | Reduction with | Reduction with Dialister | Bifidobacterium |
| Disease | | Phascolarctobacterium | Phascolarctobacterium | succinatiphilus | bifidum treatment |
| Activity | Reduction in Stool | succinatutens treatment | faecium treatment | Treatment vs Untreated | vs Untreated |
| Score (DAS) | succinate Levels | vs Untreated animals | vs Untreated animals | animals | animals |
| 1 | >15% | ✓ | | | |
| 2 | 10-15% | | ✓ | ✓ | ✓ |
| 3 | 5-10% | | | | |
| 4 | 1-5% | | | | |

| | Reduction with | | |
|---|---|---|---|
| | Dialister | Reduction with | |
| Disease | propionicifaciens | Lactobacillus | Reduction with |
| Activity | treatment vs. | intestinalis treatment | Consortia treatment |
| Score (DAS) | Untreated animals | vs Untreated animals | vs Untreated animals |
| 1 | | | ✓ |
| 2 | ✓ | ✓ | |
| 3 | | | |
| 4 | | | |

3.7. The LCMS Based Measurement of Stool Propionate of Experimental Animals Treated with Individual Strains and a Combination of Consortia.

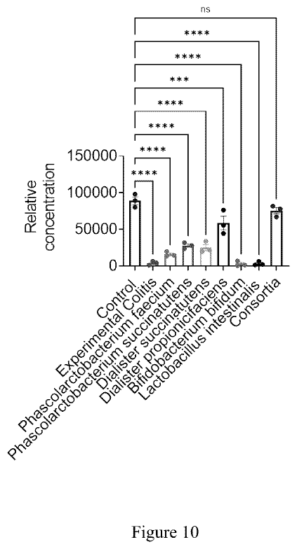

To measure propionate levels in stool using LC-MS, stool samples are first weighed and homogenized in a suitable extraction solvent, typically a mixture of methanol and water. After centrifugation to remove insoluble particles, the resulting supernatant containing soluble analytes, including propionate, was collected. The LC-MS instrument was set up with an appropriate column and mobile phase optimized for propionate separation and detection. Calibration standards covering a range of propionate concentrations were prepared and used to create a calibration curve by injecting them into the LC-MS system. Stool extracts were injected into the LC-MS system, and propionate peaks were separated and detected using mass spectrometry. Propionate in the stool sample was quantified by comparing its peak area to the calibration curve. Rigorous quality control measures were implemented to ensure the accuracy and precision of the analysis, including quality control samples and regular system suitability tests. Data analysis involves integrating propionate peaks and calculating concentrations, followed by method validation to evaluate the reliability and robustness of the procedure. Ultimately, the results were reported, providing valuable insights into stool propionate levels and their potential significance in physiological and pathological contexts. The results are depicted in FIG. 10.

TABLE 7
Shows the LCMS based measurement of stool propionate of experimental animals
treated with individual strains and a combination of consortia.
Stool Propionate Elevation

| | | Elevation with | Elevation with | Elevation with Dialister |
|---|---|---|---|---|
| Disease | | Phascolarctobacterium | Phascolarctobacterium | succinatiphilus |
| Activity | Stool Propionate | succinatutens treatment | faecium treatment vs | Treatment vs Untreated |
| Score (DAS) | Elevation Levels | vs Untreated animals | Untreated animals | animals |
| 1 | >15% | ✓ | | ✓ |
| 2 | 10-15% | | ✓ | |
| 3 | 5-10% | | | |
| 4 | 1-5% | | | |

| | Elevation with | Elevation with | | |
|---|---|---|---|---|
| | Bifidobacterium | Dialister | Elevation with | |
| Disease | bifidum treatment | propionicifaciens | Lactobacillus | Elevation with |
| Activity | vs Untreated | treatment vs. | Intestinalis treatment | Consortia treatment |
| Score (DAS) | animals | Untreated animals | vs Untreated animals | vs Untreated animals |
| 1 | | ✓ | | ✓ |
| 2 | ✓ | | ✓ | |
| 3 | | | | |
| 4 | | | | |

3.8. The Overall Disease Activity Score of Experimental Animals Treated with Individual Strains and a Combination of Consortia.

A decrease in gross pathology score for inflammation, infiltration of immune cells, ulceration, edema, and body weight body reduction score with consortia-treated animals than treated with individual strains was identified. In addition, the metabolomic profiling of stool samples of mice were also investigated. Reduction in succinate and elevation in propionate, was found to be maximum with consortia than individual strains given to the colitis animals. Furthermore, the expression of genes involved in colitis progression (IL-9, Foxo1, and SUCNR1/GPR91) in lamina propria (LP) isolated from colitis animals were significantly reduced with consortia than the individual strains.

TABLE 8
Shows the overall disease activity score of experimental animals
treated with individual strains and a combination of consortia.
Overall Disease Activity Score

| | IL-9 m-RNA | foxo1 m-RNA | | Body weight | Gross | Stool Succinate | Stool | |
|---|---|---|---|---|---|---|---|---|
| | Reduction | Reduction | SUCNR1 m-RNA | Reduction | Pathology | Reduction | Propionate | Total |
| Components | Score | Score | Reduction Score | Score | Score | Score | Elevation Score | Score |
| Phascolarctobacterium | 2 | 2 | 2 | 2 | 2 | 1 | 1 | 12 |
| succinatutens | | | | | | | | |
| Phascolarctobacterium | 2 | 2 | 2 | 2 | 2 | 2 | 2 | 14 |
| faecium | | | | | | | | |
| Dialister succinatiphilus | 1 | 1 | 1 | 2 | 1 | 2 | 1 | 9 |
| Bilidobacterium bifidum | 2 | 2 | 2 | 2 | 2 | 2 | 2 | 14 |
| Dialister | 2 | 2 | 2 | 2 | 2 | 2 | 1 | 13 |
| propionicifaciens | | | | | | | | |
| Lactobacillus | 2 | 2 | 2 | 2 | 2 | 2 | 2 | 14 |
| intestinalis | | | | | | | | |
| Consortia | 1 | 1 | 1 | 1 | 1 | 1 | 1 | 7 |

Moreover, to understand the mechanism of synergism the percent reduction in IL-9 levels were calculated with individual strains and consortia. The following formula is used to calculate percent reduction in IL-9 expression.

$$Percent of IL - 9 inhibition ( \% ) = [ {CI}^{IL - 9} - {TI}^{IL - 9} ] / {CI}^{IL - 9} * 100$$

CI^{IL-9}—Expression of IL-9 levels in Colitis animals
TI^{IL-9}—Expression of IL-9 levels in Colitis animals treated with individual strains or consortia.

| IL-9 | | % IL-9 Inhibition = |
|---|---|---|
| Expression | | [CIIL-9-TIIL-9]/ |
| (Average) | Treatment | CIIL-9 *100 |
| 78.81 | No treatment | 0% |
| 32.14 | Phascolarctobacterium faecium | 59.21% |
| 22.29 | Phascolarctobacterium | 71.70% |
| | succinatutens | |
| 22.43 | Dialister succinatutens | 71.53% |
| 35.54 | Dialister propionicifaciens | 54.90% |
| 33.77 | Bifidobacterium bifidum | 57.14% |
| 24.53 | Lactobacillus intestinalis | 68.87% |
| 9.10 | Consortia | 88.44% |

Individual bacterial strains Phascolarctobacterium faecium, Phascolarctobacterium succinatutens, Dialister succinatutens, Dialister propionicifaciens, Bifidobacterium bifidum, Lactobacillus intestinalis reduce IL-9 expression by 59.21%, 71.70%, 71.53%, 54.90%, 54.14%, and 68.87%, respectively.

When combined in a consortium, the reduction in IL-9 formation is 88.44%.

The expected additive effect (sum of individual effects) is 59.21%+71.70%+71.53%+54.90%+54.14%+68.87%, =380.35%.

The observed effect (88.44%) is less than the expected additive effect (380.35%), suggesting synergy among the bacterial strains in inhibiting IL-9 formation.

### Example 4: Profiling of Succinate Consuming and Succinate Producing Gut Microbiota Associated with Experimental Obesity

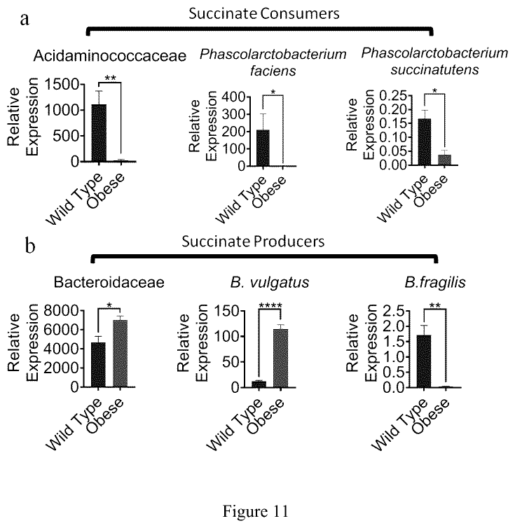

A high-fat diet (60% Fat from the Research diet) was started in C57BL/6 mice of age 8 weeks and continued till 15 weeks for the induction of experimental obesity. Once obesity is confirmed per the defined parameters, which include total body weight, fasting glucose, daily food consumption, Inflammation profile, insulin level, lipid profile, liver, and adipose tissue histological observation, their stool samples were collected in NAPG buffer and stored in −80 to −196° C. Later genomic DNA was isolated using a pure link microbiome kit, and the respective families and their members were screened using RT-PCR (FIG. 11).

### Example 5: Therapeutic Microbiota Mediated Improvement in Glucose Intolerance of DB/DB Mice (Type 2 Diabetes)

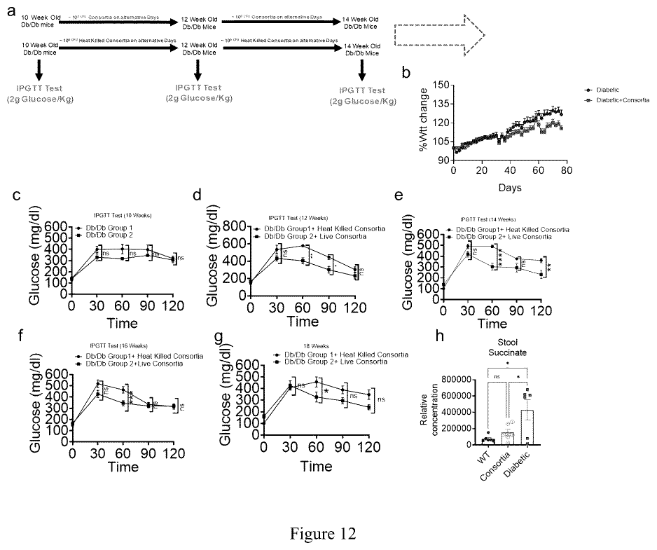

The db/db mice exhibit well-known symptoms of type 2 diabetes, such as elevated body weights, HbAlc, and glucose levels compared to the db/m controls. However, when colonized with therapeutic consortia, Db/Db mice have shown significantly lower glucose levels and body weight, indicating a slower progression of type 2 diabetes with consortia. We also measured the stool succinate levels of Db/Db mice, which were higher than those of the control animals. Furthermore, upon treatment with consortia, the succinate levels are significantly reduced (FIG. 12).

1. A combination of succinate consuming microbiotas selected from the group consisting of Phascolarctobacterium faecium, Phascolarctobacterium succinatutens, Dialister succinatutens, Dialister propionicifaciens, Bifidobacterium bifidum, and Lactobacillus intestinalis in a ratio of 1:1:1:1:1:1 or 2:4:2:4:2:4 or 4:2:4:2:4:2.

2. The combination of succinate consuming microbiotas as claimed in claim 1, wherein Phascolarctobacterium faecium is in the range of 1×10⁵ to 1×10¹⁰ CFU/ml, preferably 1×10⁷ to 1×10⁸ CFU/ml.

3. The combination of succinate consuming microbiotas as claimed in claim 1, wherein Phascolarctobacterium succinatutens is in the range of 1×10⁵ to 1×10¹⁰ CFU/ml, preferably 1×10⁷ to 1×10⁸ CFU/ml.

4. The combination of succinate consuming microbiotas as claimed in claim 1, wherein Dialister succinatutens is in the range of 1×10⁵ to 1×10¹⁰ CFU/ml, preferably 1×10⁷ to 1×10⁸ CFU/ml.

5. The combination of succinate consuming microbiotas as claimed in claim 1, wherein Dialister propionicifaciens is in the range of 1×10⁵ to 1×10¹⁰ CFU/ml, preferably 1×10⁷ to 1×10⁸ CFU/ml.

6. The combination of succinate consuming microbiotas as claimed in claim 1, wherein Bifidobacterium bifidum is in the range of 1×10⁵ to 1×10¹⁰ CFU/ml, preferably 1×10⁷ to 1×10⁸ CFU/ml.

7. The combination of succinate consuming microbiotas as claimed in claim 1, wherein Lactobacillus intestinalis is in the range of 1×10⁵ to 1×10¹⁰ CFU/ml, preferably 1×10⁷ to 1×10⁸ CFU/ml.

8. The combination of succinate consuming microbiotas as claimed in claim 1, wherein microbiotas are in the range of 1×10⁵ to 1×10¹⁰ CFU/ml, preferably 1×10⁷ to 1×10⁸ CFU/ml.

9. The combination of succinate consuming microbiotas as claimed in claim 1, wherein microbiotas are present in form of spores, in-vitro cultures, L-dried culture or freeze-dried microbial cell powder.

10. The combination of succinate consuming microbiotas as claimed in claim 1, additionally consisting of an endosymbiotic microbial consortium or an ectosymbiotic microbial consortia or combination of endosymbiotic or ectosymbiotic microbial consortia.

11. The combination of succinate consuming microbiotas as claimed in claim 1, wherein endosymbiotic microbial consortia is selected from the group comprising Firmicutes, Bacteroidetes, Actinobacteria, Proteobacteria, Fusobacteria, and Verrucomicrobia.

12. The combination of succinate consuming microbiotas as claimed in claim 1, wherein ectosymbiotic microbial consortia is selected from the group comprising Firmicutes, Bacteroidetes, Actinobacteria, Proteobacteria, Fusobacteria, and Verrucomicrobia, Malassezia and Arthrodermataceae.

13. The combination of succinate consuming microbiotas as claimed in claim 1, wherein endosymbiotic microbial consortia is in the range of 1×10⁵ to 1×10⁹ CFU/ml, preferably 1×10⁷ to 1×10⁸ CFU/ml.

14. The combination of succinate consuming microbiotas as claimed in claim 1, wherein ectosymbiotic microbial consortia is in the range of 1×10⁵ to 1×10⁹ CFU/ml, preferably 1×10⁷ to 1×10⁸ CFU/ml.

15. The combination of succinate consuming microbiotas as claimed in claim 1, additionally consisting of a mycobiota, or a virobiota or combination mycobiota or virobiota.

16. The combination of succinate consuming microbiotas as claimed in claim 1, wherein mycobiota is selected from the group comprising Candida albicans, Candida tropicalis, Candida parapsilosis, Candida glabrata, Candida krusei, Candida lusitaniae, Saccharomyces cerevisiae Penicillium commune, Aspergillus versicolor, Cryptococcus, Malassezia Globosa, Malassezia restricta, Malassezia pachydermatis, Cladosporium Herbarum, Galactomyces geotrichum, Debaryomyces hansenii, and Trichosporon.

17. The combination of succinate consuming microbiotas as claimed in claim 1, wherein virobiota is selected from the group comprising human endogenous retroviruses (HERVs), gut bacteriophages, temperate phages, microbicidal phages, Enterobacteria phage.

18. The combination of succinate consuming microbiotas as claimed in claim 1, wherein the gut bacteriophages is selected from the group comprising Caudaovirales, Alteromondales, Clostridiales.

19. The combination of succinate consuming microbiotas as claimed in claim 1, wherein the phages is selected from acetobutylicum or retroviridae family.

20. The combination of succinate consuming microbiotas as claimed in claim 1, wherein the phages infect either E. coli, or Lactobacillus, or Bacteroides uniforms or Bacteroides thetaiotaomicron or a combination thereof.

21. The combination of succinate consuming microbiotas as claimed in claim 1, wherein mycobiota is in the range 1×10⁵ to 1×10⁹ CFU/ml, preferably 1×10⁷ to 1×10⁸ CFU/ml.

22. The combination of succinate consuming microbiotas as claimed in claim 1, wherein virobiota is in the range of 1×10⁵ to 1×10⁹ CFU/ml, preferably 1×10⁷ to 1×10⁸ CFU/ml.

23. A composition comprising the combination of succinate consuming microbiotas as claimed in claim 1, along with pharmaceutically acceptable excipients that can be selected from the group comprising microcrystalline cellulose, mannitol, starch, maltodextrins, dextrins produced from non-GM sources.

24. The composition as claimed in claim 23, comprising the combination of microbiotas, present in a pharmaceutical acceptable carrier or matrix that can be selected from the group comprising kefiran, guar gum, duck feet gelatin (DFG), Dextrin, polydextrose, sago starch, Pectin, Chitosan, Chondroitin sulphate, Dextran, Cyclodextrin, Insulin, Xanthan gum, Amylose, Alginates, Shellac, Locust bean gum, methacrylic acid, methacrylic/acrylic esters, Polyvinyl acetate phthalate, Hydroxypropyl ethylcellulose phthalate, Cellulose acetate trimellitate, Cellulose acetate phthalate, hydroxypropyl methylcellulose acetate succinate (HPMCAS), and hydroxypropyl methyl-cellulose phthalate (HPMCP), azo-containing polymers and polyesters.

25. The composition as claimed in claim 23, comprising azo-containing polymers that can be selected from the group comprising azo-containing polyurethanes, azopolymeric hydrogels, copolymers of styrene, 2-hydroxyethyl methacrylate (HEMA) cross-linked with divinyl azobenzene (DVAB).

26. The composition as claimed in claim 23, comprising polyesters that can be selected from the group comprising poly D L-lactic-co-glycolic acid (PLGA), polyacrylamides, and polyvinyl alcohol (PVA).

27. The composition as claimed in claim 23, comprising the combination of microbiotas along with at least one non-digestible oligosaccharide that is selected from the group consisting of Fructooligosaccharides (FOS), Pectins or pectic polysaccharides, Mannans, Pentosans, β-glucans, arabinans or galactans, Human Milk Oligosaacarides (HMOs), Glucooligosaccharide, Xylooligosaccharide, Galactooligosaccharide, Arabinoxylan, Arabinogalactan, Galactomannan, Polydextrose, Oligofructose, Inulin and mixtures or derivatives thereof.

28. The composition as claimed in claim 23, comprising the combination of microbiotas along with at least one lactobacillus species that is selected from the group comprising: Lactobacillus acidophilus, Lactobacillus casei, Lactobacillus fermentum, Lactobacillus salivarius, Lactobacillus Intestinalis, Lactobacillus brevis, Lactobacillus leishmanii, Lactobacillus plantarum and Lactobacillus cellobiosus.

29. The composition as claimed in claim 23, wherein the combination is microencapsulated through spray-drying, freeze-drying, foam drying, or fluidized bed drying.

30. The composition as claimed in claim 23, wherein the composition is formulated for oral, intradermal, intratumoral, transdermal, parenteral, intramuscular, intrathecal, topical, intravaginal, intravesical, intracisternal, or rectal administration.

31. The composition as claimed in claim 23, wherein the composition is in the form of solid, semi-solid, lyophilized powder, liquid dosage forms, or aerosols.

32. The composition as claimed in claim 23, comprising the combination of microbiotas, present in a pharmaceutical acceptable carrier or matrix in the form of Tablets, Capsules, granules, Drops, Sachets, Liquid Suspensions, emulsions, Chewable Tablets, and Capsules with Enteric Coating etc.

33. A composition comprising the combination of succinate consuming microbiotas as claimed in claim 23, for its use in succinate reduction and for all disease associated with succinate including microbial dysbiosis that is selected from the group comprising obesity, cardiovascular disease (CVD), diabetes, Non-alcoholic fatty liver disease (NAFLD), typhoid, and paratyphoid fever, autoimmune and inflammatory diseases, psoriasis, fibrosis (Liver & Lung), cancer, intracellular infections, leaky gut associated with Alzheimer's, Parkinson, Huntington's, and other neurological disorders.
